(19) United States
(12) Patent Application Publication
(10) Pub. No.: US 2003/0158428 A1
(43) Pub. Date: Aug. 21, 2003

(54) Intermediates and processes for preparing substituted chromanol derivatives

(75) Inventors: Keith M. Devries, Chester, CT (US); Stephane Caron, Stonington, CT (US); Philip D. Hammen, East Haddam, CT (US); Geraldine P. Taber, Mystic, CT (US); John L. Tucker, Niantic, CT (US); Peter R. Rose, Ledyard, CT (US); Ronald J. Post, Mystic, CT (US); Brian K. Raymer, Cambridge, MA (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/060,731

(22) Filed: Jan. 30, 2002

(51) Int. Cl. C07D 311/74

(52) U.S. Cl. 549/401

(57) ABSTRACT

The invention relates to processes for preparing a compound of the formula

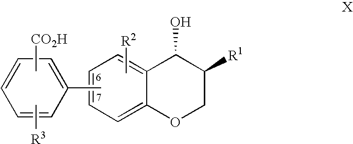

and the enantiomer of said compound, wherein the benzoic acid moiety is attached at position 6 or 7 of the chroman ring, and R¹, R², and R³ are as defined herein. The invention further relates to intermediates that are useful in the preparation of the compound of formula X above.

This invention relates to methods for preparing substituted chromanol derivatives. The substituted chromanol derivatives prepared using the methods of the present invention are disclosed in U.S. patent application Ser. No. 09/511,475, filed Feb. 23, 2000, U.S. Pat. Nos. 5,552,435 and 6,096,906, and PCT international application publication nos. WO 96/11925 (published Apr. 25, 1996), WO 96/11920 (published Apr. 25, 1996), and WO 93/15066 (published Aug. 5, 1993). Each of the foregoing United States patent applications and patents and PCT international applications are incorporated herein by reference in their entirety.

### BACKGROUND OF THE INVENTION

The substituted chromanol derivatives prepared using the methods of the present invention are effective in inhibiting the action of LTB₄, as disclosed in U.S. Pat. No. 5,552,435. As LTB₄ antagonists, the substituted chromanol are therefore useful in the treatment of LTB₄-induced illnesses such as inflammatory disorders including rheumatoid arthritis, osteoarthritis, inflammatory bowel disease, psoriasis, eczema, erythma, pruritis, acne, stroke, graft rejection, autoimmune diseases, and asthma.

The present invention provides several enhancements over the prior art methods of preparing substituted chromanol derivatives. As disclosed in Ser. No. 09/511,475, 7-halochromanol intermediates to the substituted chromanol derivatives are prepared by initial formation of an acylated chiral auxiliary which then undergoes asymmetric aldol condensation with an aromatic aldehyde, followed by reductive cleavage of the chiral auxiliary and subsequent intramolecular cyclization. For the formation of the acylated chiral auxiliary, the prior method set forth in Ser. No. 09/511,475 uses pyrophoric, air sensitive organolithium reagents such as n-butyllithium, which requires the use of cryogenic conditions. The present invention instead uses more convenient and economical reagents such as DMAP and triethylamine. Rather than a reductive cleavage of the chiral auxiliary, the present invention uses a hydrolysis to cleave the chiral auxiliary, providing a significantly higher recovery of the auxiliary and permitting much simpler isolation by crystallization. In addition, the present invention provides higher yields of the pre-cyclization intermediate using reagents that are more readily available on a commercial scale (i.e., sodium borohydride and boron trifluoride diethyl ether complex) than those required by the prior art process (lithium borohydride).

Furthermore, the present invention offers significant practical advantages in the formation of the 7-arylchromanol used as a precursor to the substituted chromanol. In particular, use of an isopropyl benzoic ester rather than a neopentyl ester to prepare a benzene boronic acid intermediate required for Suzuki cross-coupling with the 7-halochromanol was unexpectedly found to suppress the undesired formation of diisopropylamide and benzophenone (arising from condensation with a molecule of starting ester) side products observed in the method disclosed in Ser. No. 09/511,475. Transesterification of the ester is also avoided since the isopropyl ester is reacted with triisopropylborate in accordance with the new procedure described in the present disclosure. The choice of the isopropyl ester has proven to be a superior reactant in further processing since its higher stability suppresses hydrolysis to the carboxylic acid.

In addition, because of the use of the isopropyl ester and an improved choice of solvent, the present invention has the added advantage of cleaner formation of the benzene boronic acid intermediate and more facile product isolation by crystallization. The cross-coupling step of the present approach is additionally enhanced over the prior art by use of a more stable palladium phosphine catalyst and a new solvent combination which allows for preparation of substituted chromanols on a significantly larger scale. The present invention further provides improvements in methods disclosed in Ser. No. 09/511,475 for forming the 7-substituted chromanol penultimate intermediate by coupling a substituted benzene boronic acid and a substituted 7-halochromanol, rather than a substituted halobenzene and a substituted chromanol 7-boronic acid. The isopropyl ester has the added advantage of higher stability for the final ester hydrolysis step, thereby minimizing undesired premature hydrolysis.

### SUMMARY OF THE INVENTION

The present invention relates to a process of preparing a compound of formula X having the structure:

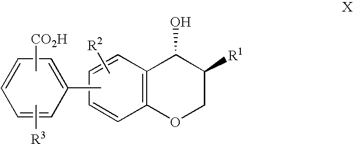

or the enantiomer of said compound, wherein in said compound of formula X the R³-substituted benzoic acid moiety is attached at carbon 6 or 7 of the chroman ring;

R¹ is —(CH₂)_{q}CHR⁵R⁶ wherein q is 0 to 4;

each R² and R³ is independently selected from the group consisting of H, fluoro, chloro, C₁-C₆ alkyl, C₁-C₆ alkoxy, phenylsulfinyl, phenylsulfonyl, and —S(O)ₙ(C₁-C₆ alkyl) wherein n is 0 to 2, and wherein said alkyl group, the alkyl moiety of said alkoxy and —S(O)ₙ(C₁-C₆ alkyl) groups, and the phenyl moiety of said phenylsulfinyl and phenylsulfonyl groups are optionally substituted by 1 to 3 fluoro groups;

R⁵ is H, C₁-C₆ alkyl, or phenyl optionally substituted by the groups set forth in the definition of R²;

R⁶ is H, C₁-C₆ alkyl, C₃-C₈ cycloalkyl, C₆-C₁₀ aryl, or 5-10 membered heteroaryl, wherein said aryl and heteroaryl groups are optionally substituted by 1 or 2 substituents independently selected from phenyl, the groups set forth in the definition of R², and phenyl substituted by 1 or 2 groups set forth in the definition of R²;

which comprises treating a compound of the formula

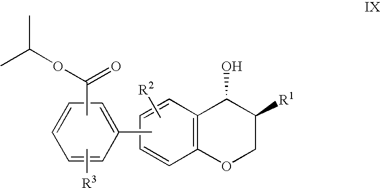

or the enantiomer of said compound of formula IX in the preparation of the enantiomer of said compound of formula X, wherein R¹, R², and R³ are as defined above, and the benzoate moiety is attached to position 6 or 7 of the chroman ring, with a base.

In said process of preparing the compound of formula X, the compound of formula IX is preferably treated with an aqueous hydroxide base, R¹ is preferably benzyl, 4-fluorobenzyl, 4-phenylbenzyl, 4-(4-fluorophenyl)benzyl, or phenethyl, R² is preferably hydrogen or fluoro, and R³ is preferably fluoro, chloro, or methyl optionally substituted by 1 to 3 fluorines. Most preferably, said compound of formula IX is treated with a base comprising aqueous lithium hydroxide, said compound of formula IX is (3S,4R)-2-(3-benzyl-4-hydroxy-chroman-7-yl)-4-trifluoromethyl-benzoic acid isopropyl ester, wherein the compound of formula X is (3S,4R)-2-(3-benzyl-4-hydroxy-chroman-7-yl)-4-trifluoromethyl-benzoic acid.

In a further aspect of the present invention, said compound of formula IX, or the enantiomer of said compound, wherein R¹, R², and R³ are as defined above, is prepared by treating a compound of the formula

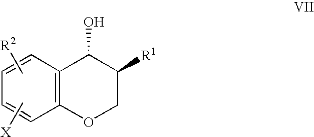

or the enantiomer of said compound of formula VII in the preparation of the enantiomer of the compound of formula IX, wherein R¹ and R² are as defined above and X is halo or C₁-C₄ perfluoroalkylsulfonate, attached at position 6 or 7 of the chroman ring, with a compound of the formula VIII:

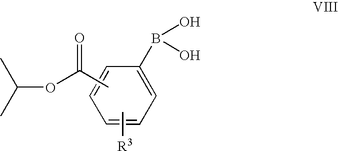

wherein R³ is as defined above, in the presence of a base or fluoride salt and a palladium catalyst.

In said process of making the compound of formula IX, or the enantiomer of said compound, preferred substituents for R¹, R², and R³ are as stated above for said process of making the compound of formula X. In another preferred embodiment, X is halo in formula VIII, the base or fluoride salt is selected from sodium carbonate, triethylamine, sodium bicarbonate, cesium carbonate, tripotassium phosphate, potassium fluoride, cesium fluoride, sodium hydroxide, barium hydroxide, and tetrabutylammonium fluoride, the palladium catalyst is selected from tetrakis(triphenylphosphine)palladium(0), dichlorobis(triphenyl-phosphine)palladium(II), pal-ladium(II) acetate, allylpalladium chloride dimer, tris(dibenzylideneacetone)dipalladium(0), and 10% palladium on carbon. Most preferably, the base or fluoride salt is potassium fluoride, the palladium catalyst is 10% palladium on carbon, the compound of formula VII is (3S,4R)-(7-bromo-3-benzyl-4-hydroxy-chroman), and the compound of formula VIII is isopropyl 4-trifluoromethyl-benzoate 2-boronic acid.

In a further aspect of the invention, the compound of formula VIII, wherein R³ is as defined above, is prepared by hydrolyzing a compound of the formula

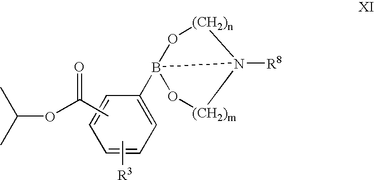

wherein R³ is as defined above, the dashed line indicates an intramolecular complex between the B and N atoms, n and m are independently 2 to 5, and R⁸ is H or C₁-C₆ alkyl. R⁸ is preferably H and preferred substituents for R³ are as stated above for said process of making a compound of formula VIII. Preferably, said hydrolysis is effected with an aqueous acid, such as hydrochloric acid, and n and m are each 2. Most preferably, said compound of formula XI is 2-[1,3,6,2]dioxazaborocan-2-yl-4-trifluoromethyl-benzoic acid isopropyl ester.

In a further aspect of the invention, a process is provided for preparing a compound of formula Xa having the structure:

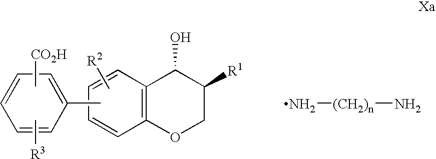

or the enantiomer of said compound, wherein R¹, R², and R³ are as defined for compound of formula X and wherein n is 2 to 4; which comprises treating a compound of formula X as set forth above, with a compound of the formula NH₂—(CH₂)ₙ—NH₂, wherein n is 2 to 4.

In the context of the present invention, the term C₁-C₈ alkyl encompasses both linear and branched chain alkyl groups, including, but not limited to, methyl, ethyl, n-propyl, isopropyl, n-butyl, sec-butyl, tert-butyl, cyclopentyl, and cyclohexyl. The alkyl group may be unsubstituted or substituted with one or more hydroxyl, halo, cyano, carboxyl, alkylacyl, arylacyl, alkoxycarbonyl, or alkylsulfoxide moieties. The term C₁-C₈ alkoxy, as used herein, encompasses ethereal moieties containing any of the C₁-C₈ alkyl groups, both substituted and unsubstituted. The term aryl, as used herein, encompasses, but not limited to, phenyl, biphenyl, naphthyl, pyridyl, indolyl, pyrazinyl, pyrimidinyl, furanyl, benzofuranyl, benzopyridyl, and thiofuranyl, and may be unsubstituted or substituted with one or more C₁-C₈ alkyl, hydroxyl, halo, cyano, carboxyl, alkylacyl, arylacyl, alkoxycarbonyl, or alkylsulfoxide moieties. The term aryloxy encompasses ethereal moieties containing any of the aryl groups noted, both unsubstituted and substituted. The terms aryl(C₁-C₈)alkyl and aryl(C₁-C₈)alkoxy encompass moieties containing any of the C₁-C₈ alkyl groups, both substituted and unsubstituted and any of the aryl groups noted, both unsubstituted and substituted. Examples of groups termed aryl(C₁-C₈)alkyl include benzyl, tolylmethyl, xylylmethyl, fluorophenylmethyl, (4-ethylphenyl)methyl, 2-(2-pyridyl)ethyl, 3-(4-hydroxyphenyl)cyclohexyl, and the like. Examples of groups termed aryl(C₁-C₈)alkoxy encompass, but are not limited to, benzyloxy, (3-tolyl)methyoxy, p-xylylmethoxy, 2-phenylethoxy, 3-phenylbutoxy, pyridylmethoxy, 3-phenyltetrahydrofuranyl, and the like.

### DETAILED DESCRIPTION OF THE INVENTION

The process of the present invention is illustrated in the following Schemes. In the discussion which follows, unless otherwise indicated, R₁, R₂, R₃, R₄, R₅, R₆, R₇, R₈, X and Y are as defined above. The following Schemes and the description which follows also apply to the enantiomeric forms of the respective compounds.

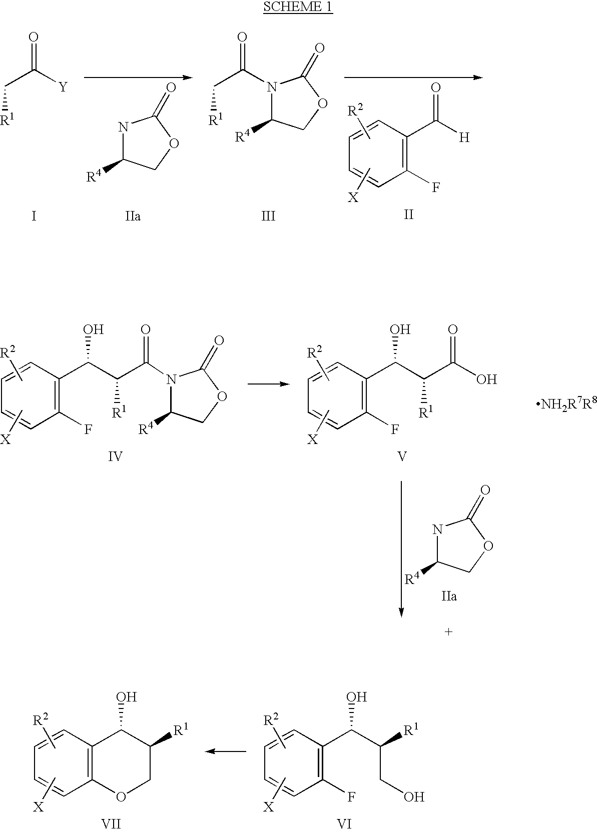

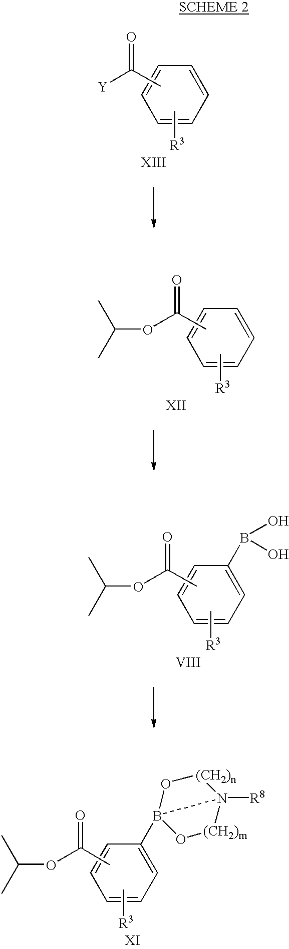

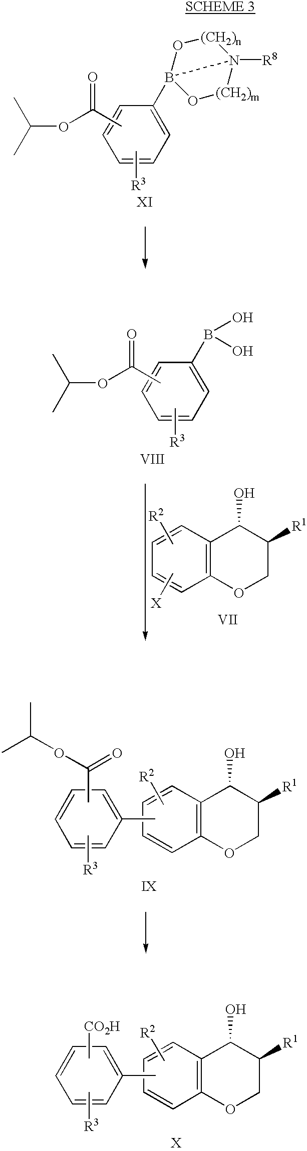

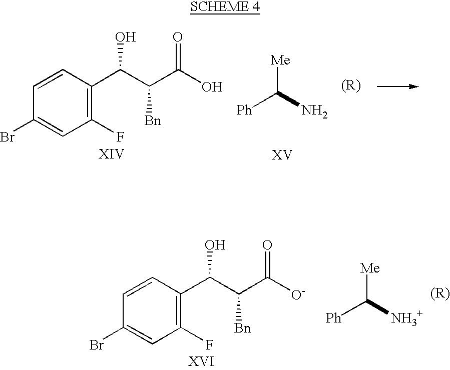

In one aspect of the invention, the compound of formula VII, or the enantiomer of said compound, wherein R¹ and R² are as defined above, is prepared by treating the diol having formula VI:

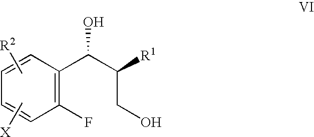

or the enantiomer of said compound of formula VI in the preparation of the enantiomer of said compound of formula VII, wherein R¹ and R² are as defined above, with a base, optionally in the presence of added copper salts.

In said process of making the compound of formula VII, or the enantiomer of said compound, preferred substituents for R¹ and R² are as stated above for said process of making the compound of formula VIII. In another preferred embodiment, the base is potassium tert-butoxide, sodium bis(trimethylsilyl)amide, potassium bis(trimethylsilyl)amide, cesium carbonate, or sodium hydride. Most preferably, the base is potassium tert-butoxide and the compound of formula VI is (1R,2S)-2-benzyl-1-(4-bromo-2-fluoro-phenyl)-propane-1,3-diol.

In a further aspect of the invention, the compound of formula VI, or the enantiomer of said compound, wherein R¹ and R² are as defined above, is prepared by treating a compound of the formula:

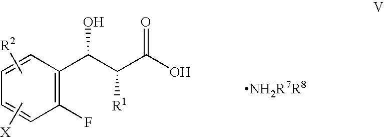

or the enantiomer of said compound of formula V in the preparation of the enantiomer of the compound of formula VI, wherein R¹, R², and X are as defined above, and R⁷ and R⁸ are independently hydrogen, C₁-C₈ alkyl, benzyl, phenyl substituted by R², C₃-C₈ cycloalkyl, or C₆-C₁₀ aryl, with a hydride reducing agent.

In said process of making the compound of formula VI, or the enantiomer of said compound, preferred substituents for R¹, R², and X are as stated above for said process of making the compound of formula VII. In another preferred embodiment, the reducing agent is sodium borohydride in the presence of boron trifluoride diethyl ether complex or boron trifluoride tetrahydrofuran complex, borane tetrahydrofuran complex, or borane dimethyl sulfide complex. Most preferably, the compound of formula V is (2R,3R)-benzylammonium-2-benzyl-3-(4-bromo-2-fluoro-phenyl)-3-hydroxy-propionic acid, and the reducing agent is sodium borohydride in the presence of boron trifluoride tetrahydrofuran complex.

In a further aspect of the invention, the compound of formula V, or the enantiomer of said compound, wherein R¹ and R² are as defined above, is prepared by treating a compound of the formula

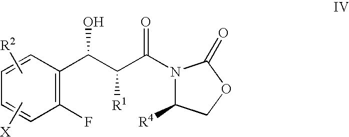

or the enantiomer of said compound of formula IV in the preparation of the enantiomer of the compound of formula V, wherein R¹, R², and X are as defined above, and R⁴ is C₁-C₈ alkyl, aryl or aryl(C₁-C₈)alkyl with a base in the presence of a peroxide, then with a reducing agent, and finally with an amine of the formula NHR⁷R⁸, where R⁷ and R⁸ are independently hydrogen, C₁-C₆ alkyl, benzyl, phenyl substituted by R², C₃-C₈ cycloalkyl, or C₆-C₁₀ aryl.

In said process of making the compound of formula V, or the enantiomer of said compound, preferred substituents for R¹, R², and X are as stated above for said process of making the compound of formula VI, and R⁴ is benzyl. In another preferred embodiment, the base is lithium hydroxide and the peroxide is aqueous hydrogen peroxide, or the base in the presence of a peroxide may be lithium hydroperoxide; the reducing agent is sodium sulfite or sodium thiosulfate, and the amine is benzylamine, dicyclohexylamine or 2-methylbenzylamine. Most preferably, the compound of formula IV is [4R-[3(2R,3R)]]-4-benzyl-3-[2-benzyl-3-(4-bromo-2-fluoro-phenyl)-3-hydroxy-propionyl]-oxazolidin-2-one, the base is lithium hydroxide, the peroxide is aqueous hydrogen peroxide, and the reducing agent is sodium sulfite, and the amine is benzylamine.

In a further aspect of the invention, the compound of formula V, or the enantiomer of said compound, wherein R¹, R² and X are as defined above, and at least one of R⁷ and R⁸ is a chiral moiety, is prepared by treating a compound of the formula

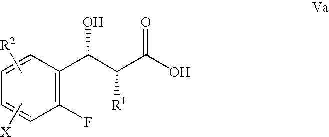

or the enantiomer of said compound of formula Va in the preparation of the enantiomer of the compound of formula V, wherein R¹, R² and X are as defined above, with a chiral amine of the formula NHR⁷R⁸, where R⁷ and R⁸ are independently hydrogen, C₁-C₆ alkyl, benzyl, phenyl substituted by R², C₃-C₈ cycloalkyl, or C₆-C₁₀ aryl, and at least one of R⁷ and R⁸ is a chira moiety.

In said process of making the compound of formula V, or the enantiomer of said compound, preferred substituents for R¹, R², and X are as stated above for said process of making the compound of formula VI. In another preferred embodiment, the compound of formula V is (2R,3R)-[R-α-methylbenzylammonium]-2-benzyl-3-(4-bromo-2-fluoro-phenyl)-3-hydroxy-propionate, and the chiral amine is R-α-methylbenzylamine.

In a further aspect of the invention, the compound of formula IV, or the enantiomer of said compound, wherein R¹, R², R⁴ and X are as defined above, is prepared by treating a compound of the formula

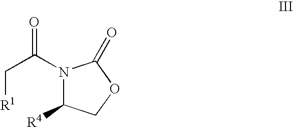

or the enantiomer of said compound of formula III in the preparation of the enantiomer of the compound of formula IV, wherein R¹ and R⁴ are as defined above with a compound of the formula II:

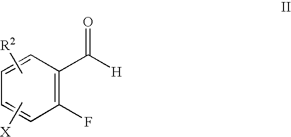

wherein R² and X are as defined above.

In said process of making the compound of formula IV, or the enantiomer of said compound, preferred substituents for R¹, R², R⁴ and X are as stated above for said process of making the compound of formula V. In another preferred embodiment, the compounds of formula II and III are treated with a titanium(IV) halide; followed by a tertiary diamine base; then a donor ligand selected from 1-methyl-2-pyrrolidinone, dimethylformamide, 1,3-dimethyl-3,4,5,6-tetrahydro-2(1H)-pyrimidinone, triethylphosphate, and 2,2′-dipyridyl; and finally a protic quench. Most preferably, the compound of formula II is 2-bromo-4-fluorobenzaldehyde, the compound of formula III is (R)-4-benzyl-3-[3-phenyl-propionyl]-oxazolidin-2-one, the titanium (IV)halide is titanium tetrachloride, the tertiary diamine base is N,N,N′,N′-tetramethlethylenediamine, the donor ligand is 1-methyl-2-pyrrolidinone, and the protic quench is aqueous ammonium chloride.

In a further aspect of the invention, the compound of formula III, or the enantiomer of said compound, wherein R¹ and R⁴ are as defined above, is prepared by treating a compound of the formula

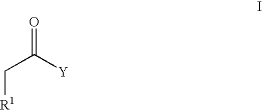

wherein R¹ is as defined above, and Y is halo or OH, with a compound of the formula

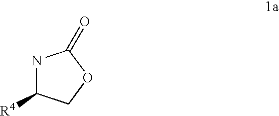

wherein R⁴ is as defined above, in the presence of a tertiary amine base and a catalytic additive.

In said process of making the compound of formula III, or the enantiomer of said compound of formula Ia in the preparation of the enantiomer of the compound of formula III preferred substituents for R¹ and R⁴ are as stated above for said process of making the compound of formula IV. In another preferred embodiment, the compounds of formula II and III are treated with a tertiary amine base selected from triethylamine and diethylisopropylamine; and the catalytic additive selected from dimethylaminopyridine and N-methylimidazole. Most preferably, the compound of formula I is 3-phenyl-propionyl chloride, the compound of formula Ia is (R)-4-benzyl-oxazolidin-2-one, R¹ is benzyl, Y is Cl, the tertiary amine base is triethylamine; and the catalytic additive is dimethylaminopyridine.

In a further aspect of the invention, the compound of formula VIII, wherein R³ is as defined above, is prepared by reacting a compound of formula XII having the structure:

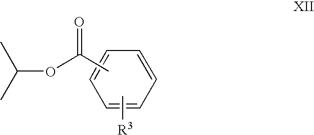

wherein R³ is as defined above, with a metal amide in the presence of a trialkylborate. In said process of preparing the compound of formula VIII, preferred substituents for R³ is as stated above for said process of preparing a compound of formula Xl; the metal amide is selected from lithium diisopropylamide or lithium 2,2,6,6-tetramethylpiperidine; and the trialkylborate is selected from triisopropylborate, triethylborate and trimethylborate. Most preferably, said compound of formula XII is (isopropoxycarbonyl)-3-trifluoromethyl-benzene, the metal amide is lithium diisopropylamide and the trialkylborate is triisopropylborate.

In a further aspect of the invention, the compound of formula XII, wherein R³ is as defined above, is prepared by treating a compound of the formula XIII having the structure:

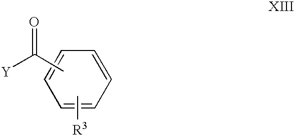

wherein R³ is as defined above and Y is OH or halo, with isopropyl alcohol and a thionyl halide. Substituents for R³ are as stated above for said process of making a compound of formula XI. Preferably, said esterification is effected using thionyl chloride or bromide. Most preferably, said compound of formula XIII is 3-trifluoromethyl-benzoyl chloride.

In accordance with the present invention, chiral auxiliary IIa is acylated in the first step of the pathway shown in Scheme 1 with an acylchloride of formula I in the presence of a tertiary amine base. The base may be triethylamine or diethylisopropylamine, and is preferably triethylamine. The reaction is favorably carried out in the presence of an additive such as dimethylaminopyridine or N-methyl imidazole, which is preferably dimethylaminopyridine, in a solvent such as dichloromethane or 1,2-dichloroethane, preferably dichloromethane, at a temperature between −20° C. and 40° C., preferably about room temperature to afford a compound of formula III.

The second step of the preparative method is a diastereoselective aldol reaction (Scheme 1). Acylated chiral auxiliary III is treated with a titanium(IV) halide, preferably titanium tetrachloride, in an aprotic solvent such as dichloromethane, 1,2-dichloroethane, or toluene, preferably dichloromethane, at a temperature of about −80 to 0° C., preferably −60 to −50° C., followed by treatment with a tertiary diamine base, such as N,N,N′,N′-tetramethlethylenediamine, preferably, at a temperature of about −80 to 0° C., preferably −65 to −50° C. This is followed by treatment with a donor ligand, such as 1-methyl-2-pyrrolidinone, dimethylformamide, 1,3-dimethyl-3,4,5,6-tetrahydro-2(1H)-pyrimidinone, triethylphosphate, or 2,2′-dipyridyl, preferably 1-methyl-2-pyrrolidinone, at a temperature of about −80 to 0° C., preferably −65 to −50° C. This mixture is treated with substituted benzaldehyde II at a temperature of about −80 to 0° C., preferably −65 to −50° C., over a period of about 2 hours, and allowed to warm to a temperature of 0 to 30° C., preferably 15° C., over a period of about one to 24 hours, preferably about 4 hours. This mixture is treated with aprotic quench, preferably aqueous ammonium chloride, at a temperature of 0 to 30° C., preferably 15° C., to yield alcohol IV. Where treatment with a donor ligand is done, the alcohol IV is, in some cases, provided as a crystalline solvate with the donor ligand. Stirring the quenched reaction mixture with a solid support such as Celite™ for a period of about 12 hours at a temperature of about 20° C. improves the filtration of the reaction mixture for removal of titanium byproducts.

The third step shown in Scheme 1 is the hydrolysis of the chiral aldol product IV to regenerate the chiral auxiliary IIa. Compound IV is treated with lithium hydroxide and aqueous hydrogen peroxide, or lithium hydroperoxide, preferably a mixture of lithium hydroxide and aqueous hydrogen peroxide, in a solvent such as tetrahydrofuran, diisopropyl ether or tert-butyl methyl ether, preferably tetrahydrofuran, at a temperature between 0° C. and 40° C., preferably about room temperature, for a period between 5 and 48 hours, preferably about 15 hours. The reaction mixture is treated with a reducing agent such as sodium sulfite or sodium thiosulfate, preferably sodium sulfite, followed by treatment with an amine such as benzylamine, dicyclohexylamine, 2-methylbenzylamine, preferably benzylamine, afford the salt V. Compound IIa can be recovered from the mother liquor and purified by extraction and crystallization. In the present invention, hydrolysis of IV serves to cleave the chiral auxiliary whereas the process of the prior art method used a reductive cleavage to provide compound VI. Among the several advantages to the present process, the recovery of the auxiliary IIa is very high and also much simpler because compound XIV (as the free acid) can be crystallized as a salt (V) while auxiliary IIa does not form a salt under the conditions used. Compounds V and IIa can be separated by a simple crystallization. In one embodiment, the formation of the benzylamine salt (V) is high yielding. The use of a chiral amine allows for enantioenrichment of compound XIV. Therefore, if a compound such as XIV is obtained in a low enantiomeric excess, the use of a chiral amine such as XV will provide a final product XVI of higher enantiomeric excess (Scheme 4). This was not possible by previous approaches where compound VI was generated directly since it did not allow for salt formation.

The fourth step in Scheme 1 is the reduction of a carboxylic acid. Compound V is treated with a reducing agent such as sodium borohydride in the presence of boron trifluoride diethyl ether complex or boron trifluoride tetrahydrofuran complex, borane tetrahydrofuran complex, or borane dimethyl sulfide complex, at a temperature between 0° C. and reflux, preferably 35-40° C., for a period between 10 and 48 hours, preferably about 29 hours. The reaction is treated with an aqueous acid such as citric acid, to provide an alcohol of formula VI.

The fifth step in Scheme 1 is an intramolecular aromatic substitution whereby the primary hydroxyl in diol VI displaces ortho fluorine to generate the chromanol ring system of VII. Diol VI is treated with a base, such as potassium tert-butoxide, sodium bis(trimethylsilyl)amide, potassium bis(trimethylsilyl)amide, or cesium carbonate, preferably potassium tert-butoxide, in an aprotic solvent such as THF, or 1-methyl-2-pyrrolidinone, preferably THF, at a temperature of between ambient temperature and 130° C., preferably about 70° C., for a period of 30 minutes to 12 hours, typically about one hours, giving chromanol VII.

Illustrated in Scheme 2 are methods to prepare the desired isopropyl ester starting material for the subsequent step, i.e., the formation of a boronic acid. In the second step of Scheme 2, isopropyl ester XII is treated with a metal amide base such as lithium diisopropylamide or lithium 2,2,6,6-tetramethylpiperidine preferably lithium diisopropylamide, in the presence of a trialkylborate such as triisopropylborate, triethylborate, or trimethylborate, preferably triisopropylborate, in an ethereal solvent such as tetrahydrofuran, diisopropyl ether, or methyl tert-butyl ether, preferably tetrahydrofuran, over a temperature range of about −40° C. to room temperature, preferably at about 0° C. After a period of 10 minutes to 5 hours, typically about 1 hour, the reaction is quenched with aqueous acid giving boronic acid VIII.

The third step in Scheme 2 is the formation of the diethanolamine complex XI of the boronic acid (VIII). This complex formation serves to facilitate the handling of boronic acid VIII before proceeding to the second step of Scheme 3, wherein the boronic acid VIII is reacted with diethanolamine in a solvent such as isopropanol, ethanol, methanol, hexanes, toluene, or a combination of the foregoing solvents, preferably isopropanol with hexanes, at a temperature within the range of 0° C. to reflux temperature, preferably ambient temperature, for a period of 15 minutes to 10 hours, preferably 10 hours, to provide the diethanolamine complex XI.

The first step in Scheme 3 is the hydrolysis of the diethanolamine complex XI to boronic acid VIII according to methods known to those skilled in the art. Such methods include the use of aqueous acid, such as hydrochloric acid in a solvent such as tetrahydrofuran, toluene, tert-butyl methyl ether, diisopropyl ether, or a mixture of the foregoing solvents, preferably a mixture of tetrahydrofuran and toluene, at a temperature between 1° C. and 60° C., preferably ambient temperature, for a period of 1 to 12 hours, preferably about 3.5 hours. After generation from XI, compound VIII can either be carried on in situ or isolated as a solid prior to the coupling with VII in step 2. Compound X is isolated as a solid by displacing the THF solvent with hexanes or some other non-polar solvent. The use of the isopropyl ester allows for the crystallization of compound IX.

The second step 2 in Scheme 3 is a Suzuki coupling between boronic acid VIII and chromanol VII to form the biaryl bound of IX. To carry out this process, a mixture is prepared containing boronic acid VIII, chromanol VII, a palladium catalyst, such as dichlorobis(triphenylphosphine)palladium(II), palladium(II) acetate, optionally in the presence of triphenylphosphine, preferably dichlorobis(triphenylphosphine)palladium(II), a base, such as sodium carbonate, sodium bicarbonate, cesium carbonate, tripotassium phosphate, or sodium hydroxide, preferably sodium carbonate, and a solvent such as toluene, ethanol, dimethoxyethane, tetrahydrofuran, or a mixture of the foregoing solvents, optionally containing water, preferably a mixture of toluene and tetrahydrofuran containing water, at a temperature of between ambient temperature and reflux temperature, preferably about 80° C., for a period of about 10 minutes to about 6 hours, preferably 2-3 hours, to provide biaryl ester IX.

The third step in Scheme 3 is an ester hydrolysis. Ester IX is treated with aqueous hydroxide base, such as aqueous lithium hydroxide, in a solvent, such as isopropyl alcohol, at a temperature between 40° C. and reflux temperature, preferably about 80° C., for a period of about one to about 24 hours, preferably about 6 hours. The reaction mixture is cooled to ambient temperature and partitioned between aqueous base and an organic solvent, such as a mixture of hexane and isopropyl ether. The aqueous solution is acidified, and the final compound X is extracted into an organic solvent such as toluene. This method of extracting compound X with organic solvents removes neutral. In a preferred embodiment of the invention, lithium hydroxide is used in the hydrolysis of IX to X.

The process shown in a preferred embodiment in Scheme 4 is diastereomeric salt formation between carboxylic acid XIV and a chiral amine. A chiral amine, such as R-α-methylbenzylamine, may be added to a solution of XIV in an organic solvent at room temperature. After a solid forms, the diastereomeric salt is isolated by filtration, or by other techniques well known in the art. Other solvent combinations, resolving agents, and temperature ranges would also be apparent to those skilled in the art. The use of chiral amines results in enantioenrichment of one antipode of intermediate Va, e.g., XIV., In addition, due to preferential reaction with one antipode of the amine, use of a racemic amine also permits enantioselection by preferential crystallization. Thus, (R)-α-methylbenzylamine forms a crystalline solid with XIV, but under similar conditions the (S)-antipode of the chiral amine does not.

The compounds prepared by the processes of the invention can be administered to humans for the treatment of LTB₄ induced illnesses, including inflammatory disorders, such as rheumatoid arthritis, osteoarthritis and inflammatory bowel disease, psoriasis, eczema, erythma, pruritis, acne, stroke, graft rejection, autoimmune diseases, and asthma, by various routes including orally, parenteraily and topically, and through the use of suppositories and enemas. On oral administration, dosage levels of about 0.5 to 1000 mg/day, advantageously about 5-500 mg/day may be given in a single dose or up to three divided doses. For intravenous administration, dosage levels are about 0.1-500 mg/day, advantageously about 1.0-100 mg/day. Intravenous administration can include a continuous drip. Variations will necessarily occur depending on the age, weight and condition of the subject being treated and the particular route of administration chosen as will be known to those skilled in the art.

The compounds prepared by the processes of the invention may be administered alone, but will generally be administered in admixture with a pharmaceutical carrier selected with regard to the intended route of administration and standard pharmaceutical practice. For example, they can be administered orally in the form of tablets containing such excipients as starch or lactose, or in capsules either alone or in admixture with excipients, or in the form of elixirs or suspensions containing flavoring or coloring agents. They can be injected parenterally, for example, intramuscularly, intravenously or subcutaneously. For parenteral administration, they are best used in the form of a sterile aqueous solution which can contain other solutes, for example, enough salt or glucose to make the solution isotonic.

The LTB₄ activity of the compounds prepared by the processes of the invention may be determined by comparing the ability of the compounds of the invention to compete with radiolabelled LTB₄ for specific LTB₄ receptor sites on guinea pig spleen membranes. Guinea pig spleen membranes were prepared as described by Chang et al. (J. Pharmacology and Experimental Therapeutics 232: 80, 1985). The ³H-LTB₄ binding assay was performed in 150 μl containing 50 mM Tris pH 7.3, 10 mM MgCl.sub.2, 9% Methanol, 0.7 nM ³H-LTB₄ (NEN, approximately 200 Ci/mmol) and 0.33 mg/ml guinea pig spleen membranes. Unlabeled LTB₄ was added at a concentration 5 μM to determine non-specific binding. Experimental compounds were added at varying concentrations to evaluate their effects on ³H-LTB₄ binding. The reactions were incubated at 4° C. for 30 minutes. Membrane bound ³H-LTB₄ was collected by filtration through glass fiber filters and the amount bound was determined by scintillation counting. The IC50 value for an experimental compound is the concentration at which 50% of specific ³H-LTB₄ binding is inhibited.

The present invention is illustrated by the following examples, but is not limited to the details thereof.

### EXAMPLE 1

(R)-4-Benzyl-3-(3-phenyl-propionyl)-oxazolidin-2-one (3)

To a solution of (R)-4-benzyl-2-oxazolidinone (30.0g, 017 mol) in a mixture of methylene chloride (250 mL), triethyl amine (47.5 mL, 0.34 mol) and 4-dimethylamino pyridine (4.15 g, 0.0034 mol) was added a solution of dihydrocinnamoyl chloride (28.1 mL, (0.19 mol) in methylene chloride (150 mL) while maintaining the temperature at approximately ambient (max 30° C.). The reaction was then stirred at ambient temperature for 2 hours and quenched into water (250 mL). The methylene chloride layer was separated and washed with 1 N HC1 (150 mL) and then aqueous sodium bicarbonate (100 mL). The methylene chloride was then removed by distillation until a volume of approximately 65 mL remained. Tetrahydrofuran (150 mL total) was added and the distillation continued until methylene chloride was completely displaced and the volume had returned to approximately 65 mL. Then hexane (120 mL) was added and the mixture stirred and the solids filtered and dried to yield 48.25 g (92.3%) of product as an off-white solid which was characterized by high performance liquid chromatography to be of high purity and identical to samples of the same compound prepared by other routes.

### EXAMPLE 2

[4R-[3(2R,3R)]]-4-benzyl-3-[2-benzyl-3-(4-bromo-2-fluoro-phenyl)-3-hydroxy-propionyl]-oxazolidin-2-one (5)

To a solution of (R)-4-benzyl-3-(3-phenyl-propionyl)-oxazolidin-2-one (100 g, 0.32 mol) in methylene chloride (1000 mL) at −50° C. to −60° C. was added a 1 M solution of titanium tetrachloride in methylene chloride (390 mL, 0.39 mol). Then while maintaining −50° C. to −55° C., tetramethyl ethylene diamine (148 mL, 0.98 mol) was added, followed by methylene chloride (100 mL) and N-methyl pyrrolidinone (62 mL, 0.65 mol). To this mixture at −50° C. to −65° C. was added a solution of 2-bromo-4-fluorobenzaldehyde (62.5 g, 0.31 mol) in methylene chloride (250 mL) over approximately 2 hours. The reaction was then warmed to approximately 15° C. and diluted with a solution of ammonium chloride (80 g, 1.48 mol) in water (500 mL). The resulting precipitate of titanium dioxide was filtered and the methylene solution was determined by HPLC and shown to contain 92% product which was held in solution for the next step.

### EXAMPLE 3

(2R 3R)-Benzylammonium-2-benzyl-3-(4-bromo-2-fluoro-phenyl)-3-hydroxy-propionic acid (6) and Recovery of (R)-4-Benzyl-2-oxazolidinone (2)

To a solution of [4R-[3(2R,3R)]]-4-benzyl-3-[2-benzyl-3-(4-bromo-2-fluoro-phenyl)-3-hydroxy-propionyl]-oxazolidin-2-one (21.36 g, 41.7 mmol) in 115 mL THF at ambient temperature was added a solution of lithium hydroperoxide (formed by mixing in order 115 mL water, 8.1 g (83.4 mmol) 35% hydrogen peroxide and 2.63 g(62.6 mmol) of lithium hydroxide monohydrate). The mixture was stirred at ambient conditions for approximately 15 hours. Residual peroxides were destroyed by addition of 2.62 g sodium sulfite and 70 mL ethyl acetate, followed by 15 mL concentrated hydrochloric acid. The organic (top) layer was separated and partially concentrated by distillation. Ethyl acetate was added and the distillation continued to remove residual water. Benzylamine (5.04 g, 45.9 mmol) was added and the mixture was stirred and then filtered. The solids were dried to give 18.7 g (97% yield) of 6, (2R,3R)-benzylammonium-2-benzyl-3-(4-bromo-2-fluoro-phenyl)-3-hydroxy-propionic acid.

(R)-4-Benzyl-2-oxazolidinone, the chiral auxiliary (2), was recovered for potential recycle from the filtrate by washing the organic solution successively with 1N NaOH (15 mL) and 1N HCl (15 mL) and concentrating the organic layer to about 20 mL followed by addition of hexanes (50 mL). The mixture was concentrated again to about 20 mL volume, hexanes (50 mL) were again added and the mixture concentrated to about 20 mL diisopropyl ether (18.5 mL) was added, followed by isopropanol (3.7 mL) and the resulting slurry was granulated, filtered and dried to give 5.48 g (74% yield) of compound (R)-4-benzyl-2-oxazolidinone (2).

### EXAMPLE 4

(1 R,2S)-2-Benzyl-1 -(4-bromo-2-fluoro-phenyl)-propane-1,3-diol (7)

To a slurry of (2R,3R)-benzylammonium-2-benzyl-3-(4-bromo-2-fluoro-phenyl)-3-hydroxy-propionic acid (50.0 g, 108 mmol) and sodium borohydride (7.14 g, 184.9 mmol) in tetrahydrofuran (250 mL) at ambient temperature was added boron trifluoride tetrahydrofuran complex (27.2 mL, 246.5 mmol). The reaction was stirred at 35-40° C. for 29 hours followed by addition of a 40% aqueous solution of citric acid (250 mL). The mixture was stirred two hours at 58° C., cooled to ambient temperature and extracted by adding methyl t-butyl ether (250 mL) and sodium chloride (20 g). The organic layer was separated and washed with a 1:1 mixture of saturated brine solution and water, followed by washes with water and saturated sodium bicarbonate until the pH was about 5.0. The organic layer was filtered through Celite™ and concentrated to a yellow oil which crystallized on standing and weighed 37.87 g (103% of theory). ¹H NMR of the product was identical to that prepared by calcium borohydride reduction of [4R-[3(2R,3R)]]-4-benzyl-3-[2-benzyl-3-(4-bromo-2-fluoro-phenyl)-3-hydroxy-propionyl]-oxazolidin-2-one (7).

### EXAMPLE 5

(3S,4R)-3-Benzyl-7-bromo-chroman-4-ol (8)

A mixture of (1R,2S)-2-benzyl-1-(4-bromo-2-fluoro-phenyl)-propane-1,3-diol (7) (33.92 g, 100 mmol) in dry tetrahydrofuran (200 mL) was cooled to 50C and potassium t-butoxide (27.76 9, 235 mmol) was added. The reaction was heated to reflux for 1 hour, cooled to 15° C. and diluted with water (80 mL). The organic layer was separated and washed with a solution of saturated sodium bicarbonate (80 mL) and then filtered through Celite™. The tetrahydrofuran was exchanged with diisopropyl ether by distillation under vacuum. The resultant slurry was cooled, granulated, filtered and dried to yield 26.0 g (81.5%) of (3S,4R)-3-benzyl-7-bromo-chroman-4-ol (8) which was identical by ¹H NMR and HPLC to an authentic sample.

### EXAMPLE 6

Preparation of IX, the Isopronyl Ester, from the Corresponding Carboxylic Acid, Method A, or the Corresponding Acid Chloride (4-trifluoromethyl benzoyl chloride), Methods B1-B2

Starting from 4-trifluoromethyl benzoic acid (commercially available):

A) Preparation of IX directly from 4-trifluoromethyl benzoic acid.

To a clean dry nitrogen purged 500 mL round bottom flask, was charged 67 mL of isopropanol, followed by 6.7 g of 4-trifluoromethyl benzoic acid. The mixture was agitated for 5 minutes and, maintaining the reaction at a temperature of less than 30° C., 6.3 g thionyl chloride was charged. After the addition was complete, the reaction was heated to reflux and stirred for 10 hrs, or until completed (<4% carboxylic acid) by LC. The reaction was then cooled to <25° C. and 40 mL hexanes added. To this organic mixture, was added a solution of sodium bicarbonate (5.4 g NaHCO₃ to 81 mL water) and the resulting quench solution stirred at <25° C. for 1 hr. The lower aqueous layer was removed and discarded. The organic layer was washed a second time with an aqueous solution of sodium bicarbonate (5.4 g NaHCO₃ to 81 mL water). The lower aqueous layer was removed and discarded. The organic layer was concentrated to an oil under reduced pressure. To the resulting oil was added 25 mL hexanes, and the solution concentrated again to an oil to effect removal of residual IPA. The oil form of IX was typically isolated in 95% yield />99% potency and was used directly in the next processing step.

B1) Preparation of 4-trifluoromethyl benzoyl chloride from 4-trifluoromethyl benzoic acid.

To a clean dry nitrogen purged 500 mL round bottom flask, was charged 6.7 g of from 4-trifluoromethyl benzoic acid, followed by 18 g of thionyl chloride. The mixture was agitated for 5 minutes then heated to reflux for 3 hrs or until complete (<2% from 4-trifluoromethyl benzoic acid by LC). Thionyl chloride was then removed by distillation under reduced pressure. The concentrated oil of 4-trifluoromethyl benzoyl chloride is used directly in the ester formation step described below.

B2) Preparation of IX from 4-trifluoromethyl benzoyl chloride.

To a clean dry nitrogen purged 500 mL round bottom flask, was charged 49 mL of isopropanol, followed by 6.9 g of 4-trifluoromethyl benzoyl chloride. The mixture was agitated for 5 minutes then was heated to 50-55° C. and stirred for 2 hrs, or until completed (<2% carboxylic acid chloride) by LC. The reaction was then cooled to <25° C. and 40 mL hexanes added. To this organic mixture, was added a solution of sodium bicarbonate (5.4 g NaHCO₃ to 81 mL water) and the resulting quench solution stirred at <25° C. for 1 hr. The lower aqueous layer was removed and discarded. The organic layer was washed a second time with an aqueous solution of sodium bicarbonate (5.4 g NaHCO₃ to 81 mL water). The lower aqueous layer was removed and discarded. The organic layer was concentrated to an oil under reduced pressure. To the resulting oil was added 25 mL hexanes, and the solution concentrated again to an oil to effect removal of residual IPA. The oil form of IX was typically isolated in 95% yield />99% potency and was used directly in the next processing step.

### EXAMPLE 7

2-[1,3,6,2]Dioxazaborocan-2-yl-4-trifluoromethyl-benzoic acid isopropyl ester (11)

A solution of lithium diisopropyl amide was made up by adding hexyl lithium (100 mL of a 2.5M solution in hexanes, 0.25 mol) to a solution of diisopropyl amine (37 mL, 0.26 mol) in 90 mL tetrahydrofuran at 0° C. The solution was then added over 40 minutes to a solution of 4-trifluoromethylbenzoic acid isopropyl ester (9) (40 g, 0.17 mol) and triisopropyl borate (80 g, 0.21 mol) in tetrahydrofuran (200 mL) at 0 ° C. The reaction was then diluted with hexanes (300 mL) followed by addition of a solution of water (230 mL) and concentrated hydrochloric acid (40 mL). The layers were separated and the organic layer was concentrated in vacuo to give 10 (an oil). The oil was dissolved in isopropanol (60 mL), followed by addition of hexanes (110 mL) and diethanolamine (18.2 g, 0.19 mol). The resulting product, 2-[1,3,6,2]dioxazaborocan-2-yl-4-trifluoromethyl-benzoic acid isopropyl ester (11), was filtered and dried to give 53.4 g (90% yield).

### EXAMPLE 8

2-(2-Methyl-ethoxycarbonyl)-5-trifluoromethyl-benzeneboronic acid (10)

To 2-[1,3,6,2]dioxazaborocan-2-yl-4-trifluoromethyl-benzoic acid isopropyl ester (11) (10.0 Kg, 29.0 mol) in a mixture of tetrahydrofuran (25 L), toluene (25 L) and water (60 L) was added concentrated hydrochloric acid (6.5 L) over about 50 minutes. The mixture was stirred for 3.5 hours and the layers were separated. To the organic layer was added hexanes (50 L) and the mixture was concentrated to about 5 L. The cycle was repeated until GC analysis of the reaction mixture showed less than 1% of tetrahydrofuran and less than 5% toluene. The resulting solid was granulated for a minimum of 2 hours and the solid was filtered and dried to provide 2-(2-methyl-ethoxycarbonyl)-5-trifluoromethyl-benzeneboronic acid (6.8 Kg, 85%).

### EXAMPLE 9

(3S,4R)-2-(3-Benzyl-4-hydroxy-chroman-7yl )-4-trifluoromethyl-benzoic acid isopropyl ester (12)

To a solution of (3S,4R)-3-benzyl-7-bromo-chroman-4-ol (8) (8.40 Kg, 26.4 mol) in a mixture of toluene (33.6 L) and tetrahydrofuran (21.0 L) was added sodium carbonate (5.67 Kg), water (33.6 L), Cl₂Pd(PPh₃)₂ (94.25 g, 134.3 mmol) and 2-(2-methyl-ethoxycarbonyl)-5-trifluoromethyl-benzene-boronic acid (8.01 Kg, 29.0 mol). The reaction mixture was stirred at 80° C. for about 2-3 hours, cooled to 40° C., filtered through a pad of Hyflo Supercel™ filter aid and washed with toluene (about 8 L). The layers of the filtrate were separated and the organic layer was concentrated to an oil which was used directly in the next step.

### EXAMPLE 10

(3S,4R)-2-(3-Benzyl-4-hydroxy-chroman-7-yl)-4-trifluoromethyl-benzoic Acid (13)

To a solution of the crude (3S,4R)-2-(3-Benzyl-4-hydroxy-chroman-7-yl)-4-trifluoromethyl-benzoic acid isopropyl ester (12) (10.5 Kg, 22.3 mol) in isopropyl alcohol (84 L) was added water (16.8 L) and lithium hydroxide monohydrate (2.8 Kg, 66 mol). The reaction was stirred at 80° C. for 6 hours, cooled to 40° C., and water (72 L) and hexanes (52.5 L) were added. The layers were separated, toluene (52.2 L) was added, and concentrated hydrochloric acid (6 L) was slowly added (pH of the aqueous layer <2). The aqueous layer was removed and the organic layer was concentrated to an oil under vacuum at a temperature below 40° C. to afford (3S,4R)-2-(3-benzyl-4-hydroxy-chroman-7-yl)-4-trifluoromethyl-benzoic acid (9.0 Kg, 95%).

### EXAMPLE 11

(2R,3R)-[R-α-Methylbenzylammonium] 2-benzyl-3-(4-bromo-2-fluoro-phenyl)-3-hydroxypropionate (XVI)

To a solution of (2R,3R)-2-benzyl-3-(4-bromo-2-fluoro-phenyl)-3-hydroxy-propionic acid (12.5 g/35.4 mmol) in ethyl acetate (62.5 ml) was added of R-α-methylbenzylamine (5.0 ml/1.1 eq.) with agitation. After adding ethyl acetate (50 ml) to mobilize, and granulating a short while, the precipitate was filtered to afford (2R,3R)-R-α-methylbenzylammonium 2-benzyl-3-(4-bromo-2-fluoro-phenyl)-3-hydroxy-propionate (13.5 g/28.5 mmol, 81%).

What is claimed is:

1. A process of preparing a compound of formula X having the structure: 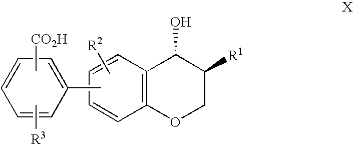
or the enantiomer of said compound, wherein R¹ is —(CH₂)_{q}CHR⁵R⁶ and q is 0 to 4;
each R² and R³ is independently selected from the group consisting of H, fluoro, chloro, C₁-C₆ alkyl, C₁-C₆ alkoxy, phenylsulfinyl, phenylsulfonyl, and —S(O)ₙ(C₁-C₆ alkyl) where n is 0 to 2, and said alkyl group, the alkyl moiety of said alkoxy and —S(O)ₙ(C₁-C₆ alkyl) groups, and the phenyl moiety of said phenylsulfinyl and phenylsulfonyl groups are substituted by 0 to 3 fluoro groups;
R⁵ is H, C₁-C₆ alkyl, or phenyl substituted by R²;
R⁶ is H, C₁-C₆ alkyl, C₃-C₈ cycloalkyl, C₆-C₁₀ aryl, or 5-10 membered heteroaryl, wherein said aryl and heteroaryl groups are substituted by 1 or 2 substituents independently selected from phenyl, the groups set forth in the definition of R², and phenyl substituted by 1 or 2 groups set forth in the definition of R²;
which comprises treating a compound of the formula IX 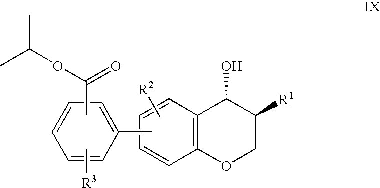
or the enantiomer of said compound of formula IX in the preparation of the enantiomer of said compound of formula X, wherein R¹, R², and R³ are as defined above, with a base.

2. The process of claim 1, wherein the base is an aqueous hydroxide base.

3. The process of claim 1, wherein R¹ is benzyl, 4-fluorobenzyl, 4-phenylbenzyl, 4-(4-fluorophenyl)benzyl, or phenethyl, R² is hydrogen or fluoro, and R³ is fluoro, chloro, or methyl substituted by 0 to 3 fluorines.

4. The process of claim 1, wherein the base is aqueous lithium hydroxide.

5. The process of claim 1, wherein said compound of formula IX is (3S,4R)-2-(3-benzyl-4-hydroxy-chroman-7-yl)-4-trifluoromethyl-benzoic acid isopropyl ester, and said compound of formula X is (3S,4R)-2-(3-benzyl-4-hydroxy-chroman-7-yl)-4-trifluoromethyl-benzoic acid.

6. The process of claim 1, wherein the compound having formula IX, or the enantiomer of the compound having formula IX, is prepared by treating a compound of the formula 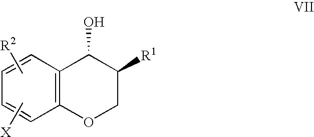
or the enantiomer of said compound of formula VII in the preparation of the enantiomer of the compound of formula IX, wherein R¹ and R² are as defined above and X is halo or C₁-C₄ perfluoroalkylsulfonate, with a compound of the formula VIII: 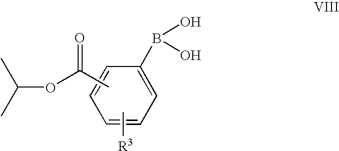
wherein R³ is as defined above, in the presence of a base or fluoride salt and a palladium catalyst.

7. The process of claim 6, wherein the compound of formula VIII is prepared by hydrolyzing a compound of the formula: 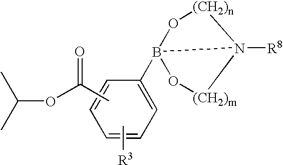
wherein R³ is as defined above, the dashed line indicates an intramolecular complex between the B and N atoms, n and m are independently 2 to 5, and R⁸ is H or C₁-C₆ alkyl.

8. A process of preparing a compound having formula IX, 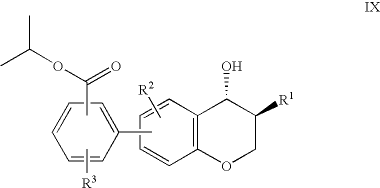
or the enantiomer of said compound, wherein R¹ is —(CH₂)_{q}CHR⁵R⁶ and q is 0 to 4;
each R² and R³ is independently selected from the group consisting of H, fluoro, chloro, C₁-C₆ alkyl, C₁-C₆ alkoxy, phenylsulfinyl, phenylsulfonyl, and —S(O)ₙ(C₁-C₆ alkyl) where n is 0 to 2, and said alkyl group, the alkyl moiety of said alkoxy and —S(O)ₙ(C₁-C₆ alkyl) groups, and the phenyl moiety of said phenylsulfinyl and phenylsulfonyl groups are substituted by 0 to 3 fluoro groups;
R⁵ is H, C₁-C₆ alkyl, or phenyl substituted by groups set forth in the definition of R²;
R⁶ is H, C₁-C₆ alkyl, C₃-C₈ cycloalkyl, C₆-C₁₀ aryl, or 5-10 membered heteroaryl, wherein said aryl and heteroaryl groups are substituted by 1 or 2 substituents independently selected from phenyl, groups set forth in the definition of R², and phenyl substituted by 1 or 2 groups set forth in the definition of R²; which comprises treating a compound of the formula 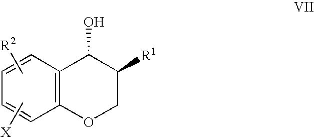
or the enantiomer of said compound of formula VII in the preparation of the enantiomer of the compound of formula IX, wherein R¹ and R² are as defined above and X is halo or C₁-C₄ perfluoroalkylsulfonate, with a compound of the formula VIII: 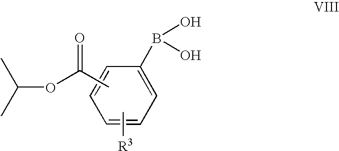
wherein R³ is as defined above, in the presence of a base or fluoride salt and a palladium catalyst.

9. The process of claim 8, wherein R¹ is benzyl, 4-fluorobenzyl, 4-phenylbenzyl, 4-(4-fluorophenyl)benzyl, or phenethyl; R² is hydrogen or fluoro; and R³ is fluoro, chloro, or methyl substituted by 0 to 3 fluorines.

10. The process of claim 8, wherein X is halo.

11. The process of claim 8, wherein the fluoride salt is potassium fluoride and the palladium catalyst is 10% palladium on carbon.

12. The process of claim 8, wherein the compound of formula VII is (3S,4R)-(7-bromo-3-benzyl-4-hydroxy-chroman), and the compound of formula VIII is isopropyl 4-trifluoromethyl-benzoate 2-boronic acid.
